(19) United States
(12) Patent Application Publication
(10) Pub. No.: US 2016/0193404 A1
(43) Pub. Date: Jul. 7, 2016

(54) APPARATUS FOR TREATMENT OF BODY CAVITIES

(71) Applicant: UNIVERSITY OF DUNDEE, Dundee (GB)

(72) Inventors: Eric ABEL, Dundee (GB); Paul WHITE, Blairgowrie (GB)

(21) Appl. No.: 14/911,614

(22) PCT Filed: Aug. 28, 2014
(86) PCT No.: PCT/GB2014/052604

§ 371 Date: Feb. 11, 2016

(30) Foreign Application Priority Data
Aug. 28, 2013 (GB) 1315290.5

(51) Int. Cl. A61M 3/02; A61M 31/00

(52) U.S. Cl. CPC A61M 3/0279; A61M 31/00; A61M 2210/0681; A61M 2210/1017

(57) ABSTRACT

An apparatus for treatment of an anatomical region comprises a primary tube, which is connectable to a fluid control system. At least one branch extends from the primary tube and is in fluid communication with the primary tube. The apparatus is insertable into a body cavity and, when inserted, at least one branch extends into an anatomical region. In use, a fluid control system is connected to the apparatus and the primary tube and at least one branch facilitate the supply of fluid to an anatomical region. The apparatus therefore can be inserted into a body cavity of a patient to allow treatment of an anatomical region, e.g. by irrigation.

### FIELD OF THE INVENTION

The invention relates to an apparatus and method for treatment, e.g. irrigation, of anatomical regions, such as body cavities, e.g. sinuses.

### BACKGROUND OF THE INVENTION

In general, body cavities can be washed out with a fluid in a process known as lavage or body cavity irrigation. The procedure is most commonly performed with saline, antibiotic or antiseptic solution and is typically used as a means of flushing out infected debris or pus from an infected body cavity. Examples of such methods include, but are not limited to, sinus irrigation, intracranial abscess irrigation, cerebral ventricular irrigation, peritoneal lavage for the treatment of peritonitis, pleural lavage for the treatment of pleural empyema, joint space lavage for the treatment of septic arthritis, bladder lavage and abscess space lavage. Typically, gentle pressure is applied in the introduction of the fluid, and the fluid is removed from internal cavities through a suction drain.

Sinuses are air-filled cavities within the skull and, in a healthy individual, any fluid and mucous in the sinuses drains into the nasal cavity and down the back of the throat. In the head, there are four pairs of paranasal sinuses, each having a right and a left side, known as the maxillary sinuses, the frontal sinuses, the ethmoid sinuses and the sphenoid sinuses.

If one or more sinus becomes blocked for a period of time, by infection or otherwise, a surgical procedure may be required to unblock that sinus and to restore an air cavity. The ailment is known generally as sinusitis. The surgical procedure, commonly an endoscopic surgical procedure, involves removing infected tissue and enlarging the opening to the affected sinus or sinuses. Following surgery there may be some bleeding and secretion of fluid from the sinus resulting in the formation of clots and crusts, which are not only uncomfortable but may also delay healing. Sinus infections may also be a symptom or side effect of other medical conditions, e.g. cystic fibrosis. In cystic fibrosis, thick mucus in the sinus can sometimes block sinus passages and lead to chronic infection. To mitigate the risk of infection, individuals are generally prescribed sinus irrigation using a saline solution in order to flush the sinuses.

Generally the saline is introduced through a nostril, for example, using a nasal douche technique. Typically saline is introduced using an aspirator. For example, see WO 2010/126586 (Aardvark Medical LLC) and US 2012/277678 (Water Pik Inc) which describe such irrigation systems for sinuses. However, it can be difficult to ensure that the saline reaches the targeted sinus region or irrigates it effectively.

Other known methods of sinus irrigation include the use of catheters. For example, see WO 2013/016275 (Cook Medical Technologies) which describes the use of a catheter with a light source for sinus irrigation and WO 2013/016056 (Acclarent Inc.) which describes the use of balloon comprised on the catheter for transnasal dilation and irrigation of a sinus. WO 2011/008987 (Reagents of the University of Minnesota) describes a catheter and probe which are configured for gradual and incremental probe extension within the nasal and sinus passageways via an uncinate progress into the maxillary sinus ostium. CN 2091683 (Wang) describes a puncturing and irrigation device for maxillary sinus.

### SUMMARY OF THE INVENTION

According to a first aspect of the invention there is provided an apparatus for treatment of an anatomical region comprising:
a primary tube connectable to a fluid control system, and
at least one branch extending from the primary tube and in fluid communication with the primary tube,

wherein the apparatus is insertable into a body cavity and, when inserted, at least one branch extends into an anatomical region.

In use, the apparatus may be inserted into a body cavity of a patient, before, during and/or after treatment on the anatomical region. For instance, the apparatus may be inserted into a body cavity after surgery. Alternatively or additionally, the apparatus may be inserted into a body cavity to allow treatment of an anatomical region, e.g. by irrigation. Alternatively or additionally, the apparatus may be inserted into a body cavity to allow targeted delivery of an active agent to the anatomical region and/or body cavity. After insertion, at least one branch may be configured to extend into an anatomical region to lodge the apparatus in position. For example, this configuration may enable the apparatus to be hooked into position in an anatomical region.

The anatomical region may comprise an anatomical chamber and/or a body cavity. The anatomical region may comprise a sinus. The anatomical region may comprise a cavity in the peritoneal space e.g. paracolic gutters and subphrenic areas, a pleural cavity, a joint cavity, a bladder and/or large abscess space. The anatomical region may comprise a cerebral ventricular space or intracranial space (e.g. an intracranial abscess space).

The apparatus may be used for treatment, irrigation and/or removal of debris from an anatomical region.

In use, a fluid control system may be connected to the primary tube to supply fluid to an anatomical region, e.g. irrigation fluid. After connection of the fluid control system, the apparatus may facilitate irrigation of an anatomical region. In use, the primary tube and/or at least one branch may provide a fluid pathway from the fluid control system to an anatomical region. The primary tube and/or at least one branch may facilitate delivery of an irrigation fluid to an anatomical region and/or body cavity. At least one branch may facilitate the treatment and/or irrigation of an anatomical region by providing a fluid pathway directly into the anatomical region. At least one branch may facilitate flushing of an anatomical region by supplying pressurised fluid directly into the anatomical region. Alternatively or additionally, the primary tube and/or at least one branch may facilitate targeted and/or precise delivery of an active agent to an anatomical region and/or body cavity.

The primary tube and/or at least one branch may facilitate removal of fluid (e.g. irrigation fluid) from an anatomical region and/or body cavity. The primary tube and/or at least one branch may facilitate removal of fluid or debris from an anatomical region and/or body cavity by suction.

After irrigation of a sinus, the fluid control system may be disconnected from the primary tube. The apparatus may be left in position in a body cavity between treatments. Multiple irrigations of a sinus may be carried out using the same apparatus. For instance, the apparatus may be left in position and multiple irrigations may be carried out upon connection and reconnection of a fluid control system, and/or the connection of different fluid control systems, at various points in time. For instance, the apparatus may be left in position in a body cavity for a period longer than 1 hour, e.g. at least about 24 hours, such as between about 24 and about 76 hours or between about 24 and about 48 hours. During this period, a fluid control system or different fluid control systems may be periodically connected to the apparatus to facilitate periodic treatment of an anatomical region.

As used herein, “irrigation” may refer to a method of flushing and/or washing a body cavity and/or treated area of a patient using a fluid. Irrigation may mitigate the risk of infection. Irrigation may promote healing of a wound.

The primary tube may define a fluid pathway for an irrigation fluid. At least one branch may define a fluid pathway for an irrigation fluid. The fluid pathway defined by at least one branch may be continuous with the fluid pathway defined by the primary tube.

The primary tube and/or at least one branch may define an aperture or a plurality of apertures. A wall of the primary tube and/or at least one branch may define an aperture or a plurality of apertures. At least one aperture may provide or define a fluid outlet. At least one aperture may provide or define a fluid inlet. At least one aperture may provide or define a fluid outlet and/or inlet into an area of the body cavity, such as an anatomical region. At least one aperture may facilitate the ejection of irrigation fluid into an anatomical region.

A plurality of apertures may be distributed along and/or around the body of the primary tube and/or at least one branch. The provision of an aperture or plurality of apertures on a wall of the primary tube and/or at least one branch may facilitate delivery of fluid to a greater volume of the anatomical region being treated and/or may enable a more uniform delivery of fluid throughout that region. For example, the provision of a plurality of apertures may enable fluid to be distributed more evenly through the anatomical region being treated.

The aperture or apertures may comprise a circular shape, or a diamond, triangular, square or other polygonal shape. The aperture or apertures may comprise a slit in a wall of at least one branch and/or the primary tube. The relatively small size of the apertures may facilitate the delivery of irrigation fluid to an anatomical region in a jet-like manner which may improve the flushing efficiency of the apparatus.

The aperture or apertures may be transformable between an open and a closed state. In the open state, the aperture or apertures may define a fluid outlet. When in the closed state, the aperture or apertures may limit and/or prevent fluid flow through the aperture or apertures. The aperture or apertures may be transformable between the open and closed states upon pressurisation of the apparatus and/or upon a pressure differential being applied across the aperture or apertures. The aperture or apertures may be in the open state when the apparatus is pressurised. For example, the aperture or apertures may be in the open state when a positive pressure is applied to the apparatus. The aperture or apertures may be in the closed state when no external pressure is applied to the apparatus. The aperture or apertures may comprise valves, e.g. slit valves. In use, upon pressurisation of the apparatus, the aperture or apertures may open and so facilitate the irrigation of an anatomical region by allowing fluid flow into an anatomical region. Upon removal of the pressure, the aperture or apertures may close, thus preventing fluid, such as mucous and/or debris from an anatomical region, entering into the apparatus through the apertures. For example, the apertures may close to prevent backflow of fluid from an anatomical region into the apparatus.

The relatively small size of the apertures may restrict the flow of fluid from the apparatus into the anatomical region. The apparatus may become pressurised when fluid is supplied to the primary tube. When fluid is supplied to the apparatus, the apparatus may become turgid. When pressurised, the rigidity of the apparatus may increase. This may assist in lodging the apparatus in position in an anatomical region and/or a body cavity as the apparatus may resist adverse deformation away from a preformed shape.

The primary tube may define an opening at both ends of the primary tube. The primary tube may comprise a closed end. The primary tube may comprise a closed end at a distal end of the primary tube. The primary tube may comprise a closable and/or sealable end. The primary tube may comprise a closable and/or sealable end at a distal end of the primary tube. The primary tube may be configured to restrict fluid flow from a distal end of the primary tube. For example, the primary tube may comprise a reduced cross-sectional area at a distal end and/or may comprise a cap configured to limit fluid flow from the apparatus.

At least one branch may define an opening at both ends of the branch. At least one branch may comprise a closed end. At least one branch may comprise a closed end at a distal end of the primary tube. At least one branch may comprise the distal end of the primary tube. At least one branch may comprise a closable and/or sealable end. At least one branch may comprise a closable and/or sealable end at a distal end of said branch. At least one branch may be configured to restrict fluid flow from a distal end of the at least one branch. For example, the at least one branch may comprise a reduced cross-sectional area at a distal end and/or may comprise a cap configured to limit fluid flow from the apparatus.

A closed or constricted end of the primary tube and/or at least one branch may facilitate ejection of fluid from at least one aperture defined by the primary tube and/or at least one branch.

At least one branch may extend from the primary tube. At least one branch may extend transversely from the primary tube. At least one branch may extend at an angle from the primary tube. For example, at least one branch may extend at a substantially 90° angle from the primary tube.

At least one branch may extend from an end of the primary tube. At least one branch may extend from an end of the primary tube continuously and/or congruously. At least one branch may be configured to extend from a distal end of the primary tube. At least one branch may extend transversely from a distal end of the primary tube. At least one branch may extend from the primary tube at an intermediate position between the distal and proximal ends of the primary tube.

The apparatus may comprise at least one primary junction region. At least one primary junction region may be defined as the location at which at least one branch extends from the primary tube and/or the location at which at least one branch meets the primary tube.

At least one primary junction region may be located at an intermediate position between the distal and proximal ends of the primary tube. At least one primary junction region may be located at the distal end of the primary tube.

At least one branch may be formed integral with the primary tube. At least one branch may be geometrically fixed with respect to the primary tube and/or at least one branch may comprise a fixed geometric arrangement with respect to the primary tube. At least one branch may be attached, or removably attachable to the primary tube. Attachment of at least one branch to the primary tube may be by adhesion, and/or by a connecting mechanism, such as a snap-fit, compression fit, interference fit, a screw-fit, a Luer® fit, a clip mechanism or the like. At least one branch may be insertable and/or attachable into a socket defined by the primary tube. The primary tube may comprise a projecting portion insertable and/or attachable into a socket defined by at least one branch.

At least one branch may comprise a tubular body. At least one branch may comprise a spherical body. At least one branch may comprise an ellipsoid or ovoid body. A body of at least one branch may define a circular or oval cross-section, or a cross-section of a polygonal shape, such as triangular, square, pentagonal, hexagonal or the like.

At least one branch may comprise a balloon. The balloon may be inflatable and deflatable. The balloon may assist in dilating or unblocking an anatomical region and/or may improve drainage from an anatomical region. For example, the balloon may assist in dilating or unblocking a sinus and/or may improve drainage from a sinus.

The apparatus may comprise at least one sub-branch extending from at least one branch. The sub-branch may be in fluid communication with at least one branch. The sub-branch may be in fluid communication with the primary tube. At least one sub-branch may define an opening at both ends of the sub-branch. At least one sub-branch may comprise a closed end. At least one sub-branch may comprise a closable and/or sealable end. At least one sub-branch may comprise a closable and/or sealable end at a distal end of said sub-branch. At least one sub-branch may be configured to restrict fluid flow from a distal end of the at least one sub-branch. For example, the at least one sub-branch may comprise a reduced cross-sectional area at a distal end and/or may comprise a cap configured to limit fluid flow from the apparatus.

The sub-branch may define an aperture or a plurality of apertures. The variations in aperture configurations as described above for at least one branch may apply to the apertures comprised on at least one sub-branch.

The apparatus may comprise at least one secondary junction region. At least one secondary junction region may be defined as the location at which at least one sub-branch extends from at least one branch and/or the location at which at least one sub-branch meets at least one branch. The apparatus may comprise a plurality of secondary junction regions.

At least one branch and/or sub-branch may be configured to extend into an anatomical region or a plurality of anatomical regions.

The apparatus may comprise a plurality of branches and/or a plurality of sub-branches. The apparatus may facilitate treatment of a wide surface area in different areas of the anatomical region. The plurality of branches may enable the treatment of a plurality of anatomical regions. For example, the apparatus may be configured to treat a plurality of anatomical regions simultaneously. For example, the apparatus may be configured to irrigate a plurality of anatomical regions simultaneously. The apparatus may comprise at least two branches. Two or more of a plurality of branches may be configured to extend into a separate or distinct anatomical region.

Where the apparatus comprises a plurality of branches and/or a plurality of sub-branches, the body of each branch and/or sub-branch may independently define the same or different cross-sectional areas. The variation in cross-sectional areas between branches and/or sub-branches may facilitate control of rate of fluid flow through the apparatus.

A plurality of branches and/or sub-branches may be configured to extend into a same anatomical region. Additionally or alternatively, a plurality of branches and/or sub-branches may be configured to extend into separate anatomical regions. Two or more of a plurality of branches and/or sub-branches may be configured to each extend into a separate anatomical region. Each branch and/or sub-branch may be independently configured to extend into an anatomical region or a plurality of anatomical regions.

A plurality of branches may extend from the same primary junction region. A plurality of branches may be co-located on the primary tube.

A plurality of sub-branches may extend from the same secondary junction region. A plurality of sub-branches may be co-located on the branch.

A plurality of branches may be geometrically arranged so as to treat a plurality of anatomical regions. The geometric arrangement of a plurality of branches may enable two or more branches to extend into a different anatomical region. The relative geometric arrangement of the branches in a plurality of branches may enable two or more branches to extend into a different anatomical region. The relative geometric arrangement of the branches may be complementary to a relative arrangement of anatomical regions, such as sinuses, in a patient. Where the apparatus comprises a plurality of branches, each branch may be located at a different primary junction region along the primary tube. At least one branch may be spaced apart from the other branches where the apparatus comprises a plurality of branches.

The apparatus may comprise a bridging portion. The primary tube and/or at least one branch may define the bridging portion. In use, the bridging portion may be configured to extend between two separate anatomical regions (e.g. the left and right nasal passages). The bridging portion may assist in lodging the apparatus in position. For example, the bridging portion may act as a hook over the left and right nasal passages.

The apparatus may comprise a biocompatible material. The primary tube and/or at least one branch may comprise a biocompatible material. The apparatus, primary tube and/or at least one branch may comprise a material selected from silicone, a plastics material such as PVC, and the like.

The apparatus may comprise a flexible material and/or may be resiliently deformable. The primary tube and/or at least one branch may comprise a flexible material. The primary tube and/or at least one branch may be resiliently deformable. A resiliently deformable material may facilitate the initial positioning of the apparatus in the body cavity and/or may assist in lodging the apparatus in position in the body cavity. The apparatus may comprise a preformed shape. The apparatus may comprise a moulded preformed shape. The apparatus may comprise a reinforcing arrangement or assembly. Such a reinforcing arrangement or assembly may confer a particular shape to the apparatus.

The apparatus may be provided in a plurality of sizes. The apparatus may be provided in one size. The size of the apparatus may be adjustable. The size of the apparatus may be reducible. The apparatus may be sized according to a patient.

The apparatus may be at least partially stored within a tubular structure. The apparatus may be stored in a compacted or compressed state, e.g. an elastically compressed state. When in a stored configuration, the apparatus may extend beyond at least one end of the tubular structure.

The tubular structure may be rigid or flexible. The tubular structure may comprise or be formed of a biocompatible material. For example the apparatus may comprise or substantially consist of a material selected from silicone, a plastics material, and the like, e.g. a surgical grade polymer.

The tubular structure may comprise a deployment tube. The deployment tube may be configured to insert the apparatus into a body cavity, e.g. a nasal cavity. The apparatus may be slidingly received and/or removed from the deployment tube. The apparatus may be deployed from the deployment tube into a body cavity upon removal of the deployment tube.

The deployment tube may define a slot. The slot may be a continuous slot extending along a length of the tubular structure. The slot may partially extend along a length of the tubular structure. The slot may be coterminous with the tubular structure. The slot may be configured to provide access to a tool to facilitate delivery and/or deployment of the apparatus from the deployment tube.

The apparatus and/or tubular structure may be disposable.

The apparatus may comprise a single piece system. The apparatus may comprise a single moulded body. At least one branch may be formed integral with the body of the primary tube.

The apparatus may comprise a modular system. The primary tube and at least one branch may comprise a first and second component of the modular system. At least one sub-branch may comprise a third component of the modular system.

In use, a modular construction may allow the apparatus to be tailored for use on a particular patient. For example, the length of the primary tube and/or at least one branch may be selected to enable effective treatments of different patients. Different lengths and/or sizes of the primary tube and/or at least one branch may be selected for different patients and/or according to the anatomical regions undergoing treatment.

The primary tube may be connectable to at least one branch via a connector. The primary tube may be interchangeably connectable with at least one branch. At least one branch may be connectable to at least one sub-branch via a connector. At least one branch may be interchangeably connectable to at least one sub-branch. The primary tube, at least one branch and/or at least one sub-branch may be prefitted with a connector. The connector may comprise a snap-fit, compression fit, interference fit, a screw-fit, a Luer® fit, a clip mechanism or the like.

The apparatus may comprise a connector to allow connection of the primary tube to a fluid control system. The primary tube may comprise the connector at a proximal end of the primary tube. The primary tube may be removably attachable to a fluid control system via the connector.

The connector may be configured to facilitate connection of a proximal end of the tube to a fluid control system. The connector may comprise a snap-fit, compression fit, interference fit, a screw-fit mechanism, a clip or the like. The connector may comprise a Luer® fitting.

The fluid control system may comprise a fluid delivery system. The fluid control system may be configured to supply fluid to the apparatus and/or a body cavity. Additionally or alternatively, the fluid control system may comprise a fluid removal system. The fluid control system may be configured to facilitate removal of fluid from the apparatus and/or a body cavity.

The fluid control system may comprise a container for holding fluid. The fluid control system may comprise a fluid reservoir. The fluid control system may comprise means for introducing fluid to the apparatus under pressure. For example, fluid may be introduced under pressure due to the application of pressure and/or the force of gravity. The fluid control system may comprise means for introducing and/or maintaining a positive pressure within the apparatus. The fluid control system may comprise an aspirator, a syringe, a deformable container, or the like. The fluid control system may comprise a pump. For example, the fluid control system may comprise a mechanical or electrical pump.

The fluid control system may comprise means for facilitating the removal of fluid from the apparatus and/or body cavity by suction and/or by providing a drainage pathway for the fluid. The fluid control system may comprise means for introducing and/or maintaining a negative pressure within the apparatus.

The fluid control system may comprise a pressure controller. The pressure controller may control the pressure supplied to the apparatus. For example, in use, the pressure controller may maintain a predetermined pressure within the apparatus and/or a predetermined pressure supplied to the apparatus. The pressure controller may comprise a relief valve. The pressure controller may comprise a pressure limiter. The pressure limiter may prevent the pressure applied to the apparatus exceeding a predetermined value. The pressure limiter may prevent the pressurisation of the apparatus above a predetermined value. The pressure controller may allow some leakage of fluid to prevent the pressure exceeding a predetermined value. The fluid control system may comprise a collection chamber to collect fluid leaked from the pressure controller and/or the apparatus. The collection chamber may be held at atmospheric pressure.

The apparatus may facilitate circulation of fluid through and/or via an anatomical region or a plurality of anatomical regions. The apparatus may comprise means for circulating fluid through and/or via an anatomical region.

The apparatus may comprise a first primary tube and a second primary tube. The first primary tube may be connectable to a fluid delivery system. The second primary tube may be connectable to a fluid removal system.

The first and second primary tubes may be insertable into a body cavity simultaneously or sequentially. The first and second primary tubes may be positionable adjacent to one another in a body cavity. The first and second primary tubes may be alignable with one another in a body cavity.

The first and second primary tubes may be attached or attachable to one another. For instance, the first and second primary tubes may be attachable by adhesion, or connecting materials, such as clips or the like.

The first and second primary tubes may each independently comprise at least one branch which, when the apparatus is inserted into a body cavity, extends into an anatomical region.

At least one branch extending from the first primary tube may supply fluid to an anatomical region. At least one branch extending from the second primary tube may provide a pathway for fluid removal from the anatomical region. For example, the fluid may be removed by drainage and/or by suction.

The geometry defined by the second primary tube and an associated at least one branch may substantially correspond to that of the first primary tube and an associated at least one branch. For example, the second primary tube and at least one branch extending therefrom may comprise a matching and/or corresponding profile to that of the first primary tube and at least one branch extending from the first primary tube.

In use, a fluid delivery system may be connected to the first primary tube. The fluid delivery system may supply fluid to an anatomical region via the first primary tube which is in fluid communication with at least one branch extending into the anatomical region. Fluid and/or debris may be removed from the anatomical region via at least one branch which is in fluid communication with the second primary tube. For example, the fluid and/or debris may be removed from the anatomical region when the second primary tube is connected to a fluid removal system.

The apparatus may be used for sinus treatment. The body cavity may be a nasal cavity. The anatomical region may be a sinus.

The apparatus may be insertable into a nasal cavity. When inserted, at least one branch may extend into a sinus.

At least one branch and/or sub-branch may be configured to extend into a sinus or a plurality of sinuses.

The apparatus may be configured to treat a plurality of sinuses simultaneously. For example, the apparatus may be configured to irrigate a plurality of sinuses simultaneously.

At least one branch may be configured to extend into a frontal sinus of a patient. At least one branch may be configured to extend into a maxillary sinus. For example, the apparatus may comprise at least one branch configured to extend into a frontal sinus and at least one branch configured to extend into a maxillary sinus. For example, a frontal and a maxilliary sinus may represent separate anatomical regions.

The apparatus may be configured to facilitate unilateral irrigation of a sinus or plurality of sinuses on one side of a patient. For instance, the apparatus may be configured to facilitate irrigation of a left sinus or a plurality of sinuses on a left side of a patient. Alternatively, the apparatus may be configured to facilitate irrigation of a right sinus or a plurality of sinuses on a right side of a patient.

The apparatus may be configured to facilitate bilateral irrigation of a plurality of sinuses. The apparatus may be configured to facilitate irrigation of left and right sinuses simultaneously. The apparatus may comprise at least one branch configured to extend into a left sinus and at least one branch configured to extend into a right sinus of a patient.

The apparatus may be left in position in a nasal cavity between treatments. Multiple irrigations of a sinus may be carried out using the same apparatus. For instance, the apparatus may be left in position in a nasal cavity and multiple irrigations may be carried out upon connection and reconnection of a fluid control system, and/or the connection of different fluid control systems, at various points in time. For instance, the apparatus may be left in position in a nasal cavity for a period longer than 1 hour, e.g. at least about 24 hours, such as between about 24 and about 76 hours or between about 24 and about 48 hours. During this period, a fluid control system or different fluid control systems may be periodically connected to the apparatus to facilitate periodic treatment of a sinus.

The apparatus may be configured to be insertable into a body cavity via a catheter. The apparatus may be configured to be deployed laparoscopically after entry into a body cavity.

The apparatus may be used in treatment of peritonitis, e.g. by peritoneal lavage or peritoneal irrigation. At least one branch may be configured to extend into a paracolic gutter. At least one branch may be configured to extend into a subphrenic area. The apparatus may be configured to be insertable into a body cavity through an anterior abdominal wall. The apparatus may be configured to be deployed laparoscopically after entry into a peritoneal cavity. In use, fluid, such as saline or peritoneal dialysis fluid, may be delivered to a peritoneal cavity via the apparatus and, optionally, the fluid may be removed by draining and/or via a suction tube. In use, the fluid may be removed from a suction tube located in a paracolic gutter.

The apparatus may be used in the treatment of pleural empyema or a lung abscess, e.g. by pleural cavity lavage. The apparatus may be used in the pleural cavity lavage of mammals e.g. humans, horses and dogs.

The apparatus may be used in the treatment of a joint cavity, e.g. by surgical lavage. The apparatus may facilitate treatment of a plurality of different recesses in a joint cavity. The apparatus may be used in the treatment of septic arthritis.

Septic arthritis refers to the infection of a joint cavity with microorganisms, typically bacteria, which can initiate a process of inflammation and cause irreversible damage to a joint cavity. Septic arthritis may be treated by serial needle aspiration (introducing a needle in the joint to aspirate the inflammatory liquid), and/or surgical lavage (opening and cleaning the joint cavity under anesthesia).

The apparatus may be used in the treatment of intracranial abscesses. The dura mater is a thick membrane that is outermost of the three layers of meninges that surround the brain and spinal cord. Intracranial abscesses may form above and below the dura mater. For example, an epidural abscess may form between a skull and dura mater. A subdural abscess may form between the dura mater and an underlying layer (e.g. the arachnoid mater layer). The apparatus may be inserted intracranially to allow treatment of an intracranial abscess, such as an epidural or subdural abscess. After insertion, the apparatus may allow treatment of an intracranial abscess region, e.g. by irrigation.

The apparatus may be used in the treatment of a cerebral ventricular region. For instance, the apparatus may be used to treat a patient at risk of or suffering from a haemorrhage. For example, following increased intracranial pressure or a stroke. The apparatus may be inserted into the cerebral ventricular region. After insertion, the apparatus may allow treatment of cerebral ventricular region, e.g. by irrigation. Alternatively or additionally, the apparatus may be inserted to allow targeted delivery of an active agent to the cerebral ventricular region. For example, the apparatus may allow the targeted delivery of a thrombolytic agent to the cerebral ventricular region.

Such an apparatus may comprise a plurality of apertures in the side walls of the primary tube and/or at least one branch. The apparatus may facilitate an effective delivery of fluid (e.g. irrigation fluid) and/or medication (e.g. thrombolytic agent) to the cerebral ventricular region.

According to a second aspect of the invention there is provided an apparatus for treatment of an anatomical region comprising:
a primary tube,
at least one branch extending from the primary tube, and
a fluid control system connectable to the primary tube,
the primary tube being insertable into a body cavity and, when inserted, at least one branch extends into an anatomical region.

According to a third aspect of the invention there is provided a kit for treatment of an anatomical region comprising the apparatus according to either of the first or second aspects.

The kit may comprise a modular apparatus. The kit may comprise a plurality of fluid control systems. The kit may comprise at least one dose of irrigation fluid. The fluid control system may comprise a single dose of irrigation fluid or a plurality of doses. The kit may be sterile. The kit may comprise sterile packaging. The kit may be hermetically sealed.

The kit may comprise a plurality of primary tubes. The plurality of primary tubes may comprise different sizes, lengths and/or cross-sectional areas. The kit may comprise a plurality of branches of different sizes, lengths and/or cross-sectional areas. The plurality of branches may be configured for interchangeable attachment and/or interchangeable connection to the plurality of primary tubes. The kit may comprise a plurality of connectors to allow connection of at least one primary tube with at least one branch.

The kit may comprise a cutting device. The cutting device may allow a user to cut a length of a primary tube and/or at least one branch according to a patient, e.g. by sectioning off a length of primary tube and/or a length of at least one branch according to a required size. The kit may comprise a sealing device for sealing an end of a primary tube and/or at least one branch. The sealing device may comprise a crimper, heat and/or pressure welder or the like.

According to a fourth aspect of the invention there is provided a method for treatment of an anatomical region comprising:

inserting an apparatus into a body cavity of a patient, the apparatus comprising a primary tube, wherein the primary tube is in fluid communication with at least one branch configured to extend into an anatomical region,

connecting a fluid control system to the apparatus,

introducing fluid to the anatomical region and/or removing fluid from the anatomical region, wherein the apparatus provides a fluid pathway between the fluid control system and the anatomical region.

In use, the method of treatment of an anatomical region may be carried out after surgery. The method may comprise a method for irrigating an anatomical region. The method may comprise introducing fluid to an anatomical region using a fluid control system, e.g. a fluid delivery system. The method may comprise introducing fluid to an anatomical region under pressure using the fluid control system. For example, the method may comprise pumping fluid into the anatomical region under pressure.

Additionally or alternatively, the method may comprise removing fluid from an anatomical region using a fluid control system, e.g. a fluid removal system. For instance, the method may comprise removing fluid from the anatomical region using suction. The method may comprise removing fluid and debris from an anatomical region.

Inserting the apparatus may comprise inserting through an orifice and/or via a catheter. The apparatus may be inserted laparoscopically.

The method may comprise inserting the apparatus into a body cavity of a patient using a deployment tube. The method may comprise slidingly deploying the apparatus from a deployment tube. The method may comprise inserting a deployment tube into a body cavity. The apparatus may be stored within the deployment tube. Alternatively, the method may comprise feeding the apparatus through the inserted deployment tube. Once the deployment tube is positioned in the cavity, the method may comprise deploying the apparatus and/or removing the deployment tube. The deployment tube may be removed by moving the tube proximally over the apparatus. Removal of the deployment tube may cause deployment of the apparatus.

The method may comprise supplying fluid to the apparatus prior to, during and/or after inserting the apparatus into a body cavity of a patient. The method may comprise pumping fluid into the apparatus during and/or after insertion. For example, the method may comprise supplying fluid to the apparatus to pressurise the apparatus. The apparatus is configured such that a back pressure may be maintained. When pressurised, the apparatus may comprise a turgid shape. The apparatus may resist adverse deformation when pressurised. The apparatus may maintain a preformed shape when pressurised. Such a configuration may assist in lodging the apparatus in position during insertion into a body cavity.

The fluid control system may deliver a fluid (e.g. an irrigation fluid) to an anatomical region. The fluid may deliver an active agent to an anatomical region, treat an infected anatomical region and/or mitigate the risk of infection of an anatomical region and/or may facilitate the removal of debris from an anatomical region.

The fluid may comprise a solution or dispersion comprising an active agent, or a plurality of active agents. For example, the fluid may comprise an antimicrobial agent, such as an antibacterial, antiviral and/or antifungal agent. The fluid may comprise a steroidal agent. The fluid may comprise a medicament, e.g. a medicament suitable for the treatment of cystic fibrosis and/or a thrombolytic agent. The fluid may have an antiseptic effect. The fluid may comprise a saline solution or water, e.g. distilled water.

After treatment of an anatomical region, the method may comprise disconnecting the fluid control system from the apparatus. At a later stage, the same fluid control system may be reconnected to the apparatus, or a different fluid control system may be connected to the apparatus. The apparatus may be left in position in the body cavity of a patient between treatments. The method may comprise treating the anatomical region at regular intervals.

The apparatus may be removed from the body cavity of a patient after treatment of the anatomical region is no longer required.

The method may comprise inserting an apparatus comprising a plurality of branches into a body cavity of a patient. The method may comprise treating a plurality of anatomical regions simultaneously.

The method may comprise adjusting the size of the apparatus. The method may comprise adjusting the size of the apparatus according to a patient's anatomy and/or to complement a patient's anatomy. The method may comprise reducing the size of the apparatus. For instance, for a child, the method may comprise shortening the primary tube and/or at least one branch of the apparatus. The method may comprise cutting and/or trimming portions of the apparatus prior to insertion into a patient. The method may comprise sealing an end of the primary tube and/or an end of at least one branch. The method may comprise assembling the apparatus. The method may comprise connecting and/or attaching at least one branch to the primary tube prior to insertion of the apparatus into a patient.

As used herein, a patient may refer to any mammalian subject having an anatomical region. For example, a patient may refer to a human subject. A patient may be an individual suffering from an ailment requiring treatment of an anatomical region. The ailment may be, for example, sinusitis or other ailment relating to, associated with, or proximate to a sinus, peritonitis, pleural empyema, a lung abscess, septic arthritis, an ailment in a cerebral ventricular space, such as haemorrhage, or an intracranial abscess.

According to a fourth aspect of the invention there is provided an apparatus for sinus treatment comprising:

a primary tube connectable to a fluid control system, and

at least one branch extending from the primary tube and in fluid communication with the primary tube,

wherein the apparatus is insertable into a nasal cavity and, when inserted, at least one branch extends into a sinus.

According to a fifth aspect of the invention there is provided a method for sinus treatment comprising:

inserting an apparatus into a nasal cavity of a patient, the apparatus comprising a primary tube, wherein the primary tube is in fluid communication with at least one branch configured to extend into a sinus,

connecting a fluid control system to he apparatus,

introducing fluid to the sinus and/or removing fluid from the sinus, wherein the apparatus provides a fluid pathway between the fluid control system and the sinus.

In use, the method of sinus treatment may be carried out after surgery. For example, after surgery to unblock a sinus or surgery on an area of the body proximate to, and/or associated with, a sinus. The method may comprise a method for irrigating a sinus.

According to a sixth aspect of the invention there is provided an apparatus for treatment of an anatomical region comprising:

a primary tube connectable to a fluid control system, the primary tube defining a plurality of apertures along a body of the primary tube,

wherein the apparatus is insertable into a body cavity.

It will be appreciated that the features described in detail for the first, second, third, fourth, fifth and sixth aspects of the invention may be equally applied to any of the other aspects of the invention.

### BRIEF DESCRIPTION OF DRAWINGS

These and other aspects of the present invention will now be described, by way of example only, with reference to the accompanying drawings, in which:

FIG. 1 shows the location of the paranasal sinuses in a human;

FIG. 2 shows an apparatus for sinus irrigation according to one embodiment of the present invention positioned in a patient and connected to a fluid control system;

FIG. 3 is an enlarged view of the region 3 on FIG. 2 and shows the flow of irrigation fluid into the sinuses;

FIG. 4 shows the apparatus of FIG. 2 in isolation;

FIG. 5a shows a branch of the apparatus of FIG. 2;

FIG. 5b shows a branch of an apparatus according to a further embodiment of the present invention;

FIG. 6 shows an apparatus for sinus irrigation according to another embodiment of the present invention;

FIG. 7 shows an apparatus for sinus irrigation according to another embodiment of the present invention;

FIG. 8 shows an apparatus for sinus irrigation according to another embodiment of the present invention; and

FIG. 9 shows an apparatus for sinus irrigation according to another embodiment of the present invention;

FIG. 10 shows an apparatus for peritoneal lavage according to another embodiment of the present invention;

FIG. 11 shows an individual undergoing peritoneal lavage treatment;

FIG. 12 shows an apparatus for treatment of a subdural abscess according to an embodiment of the invention;

FIG. 13 shows a side view of a deployment tube for use with embodiments of the present invention;

FIG. 14 shows a cross-sectional view along A-A of the deployment tube of FIG. 13;

FIG. 15 shows a cut-away view of an apparatus stored in a deployment tube according to one embodiment of the present invention.

### DETAILED DESCRIPTION OF THE DRAWINGS

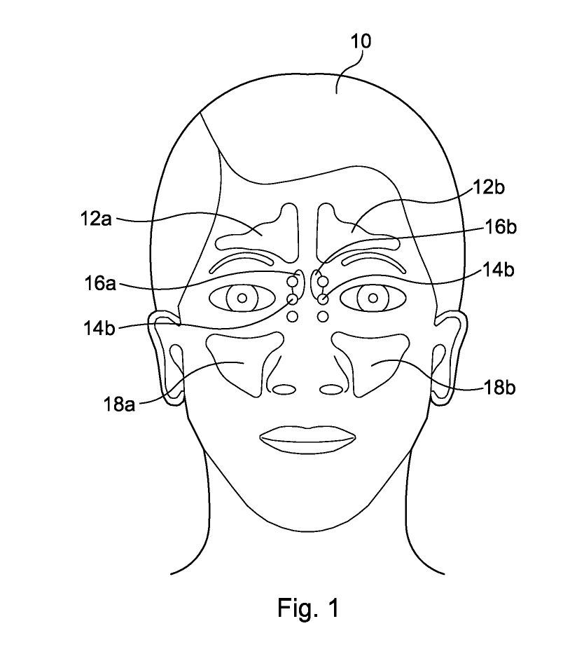

The four pairs of paranasal sinuses in the head of a person 10 are shown schematically on FIG. 1. These are the frontal sinuses (12a, 12b), the sphenoid sinuses (14a, 14b), the ethmoid sinuses (16a, 16b) and the maxillary sinuses (18b, 18b). Irrigation of one or more of these sinuses may be required, for example, after surgery to unblock a sinus. As described in detail below, apparatus according to embodiments of the present invention may be positioned in the nasal cavity to facilitate irrigation of a sinus.

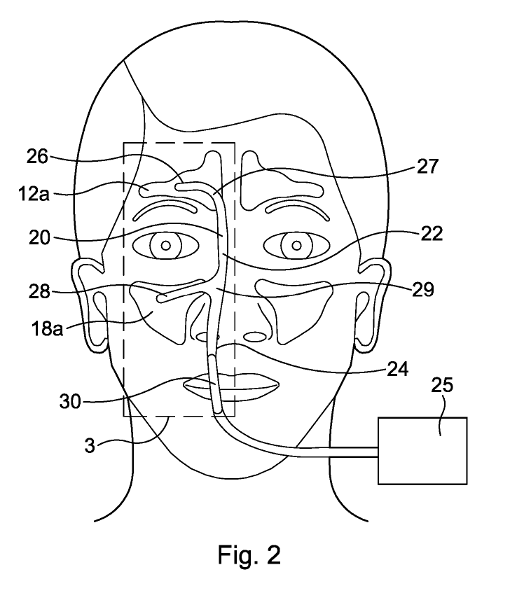

One embodiment of such an apparatus, generally identified by reference numeral 20, is shown in FIGS. 2, 3 and 4.

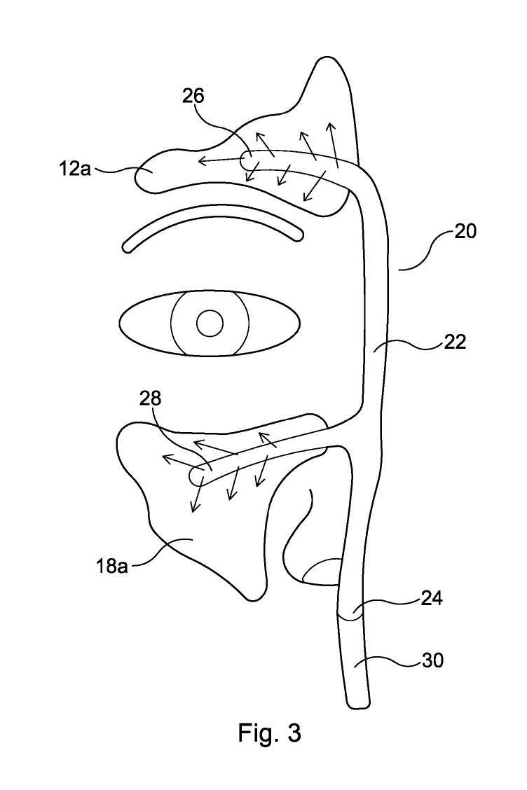

In FIG. 2, the apparatus 20 is shown after it has been inserted into a nasal cavity of a patient. The apparatus 20 comprises a primary tube 22 which is connectable to a fluid control system 25 at a proximal end 24 of the primary tube. After connection of the fluid control system 25 to the apparatus 20, fluid can be delivered via the apparatus directly to the sinus to irrigate the sinus. The mode of operation is later described in more detail, with particular reference to FIG. 3.

In the embodiment shown in FIG. 2, the fluid control system 25 is connected to the proximal end 24 of the primary tube 22 by a connector 30. In this embodiment, the connector 30 comprises a Luer® fitting, but it will be appreciated that alternative connectors may be used in other embodiments, for example, a compression fit, a snap-fit, interference fit, screw-fit mechanism or the like.

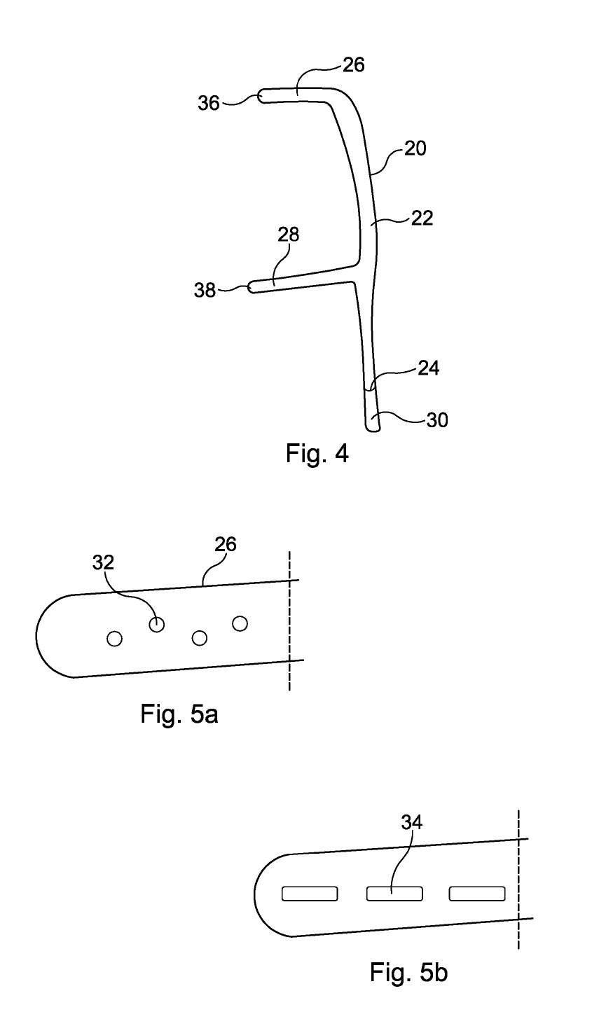

The fluid control system 25 is removably attachable to the apparatus 20. In FIG. 2, the fluid control system 25 is shown connected to the apparatus 20. However, in FIGS. 3 and 4 the fluid control system 25 has been disconnected from the apparatus 20.

The fluid control system 25 comprises a fluid reservoir and means for introducing fluid from the fluid reservoir to the apparatus 20 under pressure. The fluid control system 25 also comprises means for facilitating the removal of fluid from the apparatus and nasal cavity by suction, and/or by providing a drainage pathway for the fluid. In this embodiment, the fluid control system comprises both a fluid delivery system and a fluid removal system. In the embodiment shown in FIG. 2, the fluid control system 25 comprises a syringe. However, it will be appreciated that alternative fluid control systems may be used in different embodiments. Examples of such alternative fluid control systems include an aspirator, a deformable container, such as a squeezy bottle, a pump, or the like.

In the apparatus 20 shown in FIG. 2, two branches (26,28) extend from the body of the primary tube 22. Branch 28 extends from the primary tube 22 at primary junction region 29 which is located at an intermediate position between the distal and proximal ends of the primary tube 22. Branch 26 extends from the primary tube 22 at primary junction region 27 which is located at the distal end of the primary tube 22.

When the apparatus 20 is inserted into a nasal cavity of a patient, branch 26 extends into the right frontal sinus 12a and branch 28 extends into the right maxillary sinus 18a. After insertion, the branches (26,28) extend into the respective sinuses (12a, 18a). This configuration assists in lodging the apparatus 20 in position. For example, the configuration may enable the apparatus 20 to be hooked into position in the sinuses (12a, 18a). During a method for sinus treatment, this configuration can allow the simultaneous treatment of a plurality of sinuses. For example, in the embodiment shown in FIGS. 2-4, the apparatus can facilitate the simultaneous treatment of both the frontal sinus 12a and the maxillary sinus 18a.

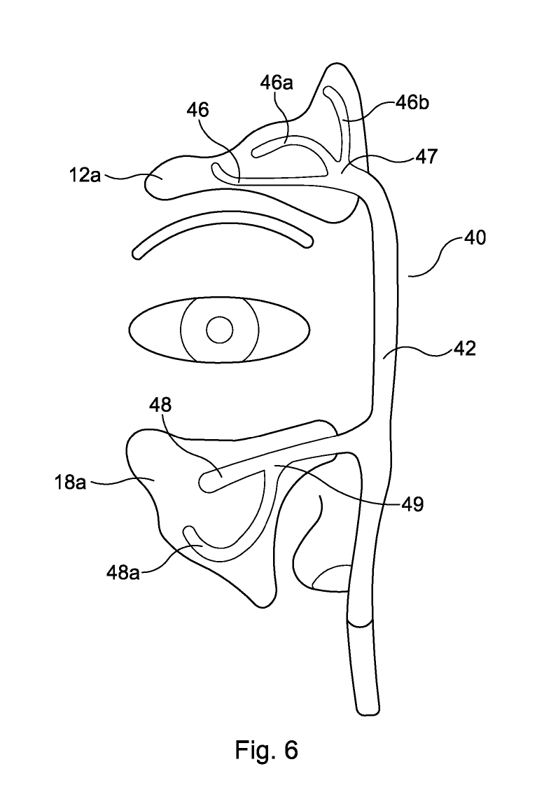

It will be appreciated that other embodiments of the present invention comprise different branch configurations, some of which are shown in FIGS. 6-9 and are described in detail below.

Each of the branches (26,28) comprises a tubular body which defines a fluid conduit. The primary tube 22 is in fluid communication with each of the branches (26,28). In the embodiment shown in FIGS. 2-4, primary tube 22 and branch 26 define a fluid pathway from the fluid control system 25 to the right frontal sinus 12a and primary tube 22 and branch 28 define a fluid pathway for fluid from the fluid control system 25 to the right maxillary sinus 18a. The branches (26,28) facilitate the delivery of irrigation fluid directly to the respective sinuses and can enable targeted irrigation of the sinuses. Additionally, or alternatively, the branches (26,28) facilitate the delivery of an active agent directly to the respective sinuses and can enable targeted treatment of the sinuses. For example, the branches can facilitate the delivery of one or more active agents selected from an antimicrobial agent, such as an antibacterial, antiviral and/or antifungal agent, a steroidal agent and a medicament, e.g. a medicament suitable for the treatment of cystic fibrosis.

The branches (26,28) are formed integral with the body of the primary tube 22. However, it will be appreciated that in other embodiments, the branches can be attached to the body of the primary tube by connecting materials such as clips or the like, or by adhesion.

The apparatus 20 comprises a biocompatible material. For example the apparatus 20 may comprise or substantially consist of a material selected from silicone, a plastics material, e.g. PVC, and the like.

The apparatus 20 comprises a resiliently deformable material. A resiliently deformable material can facilitate the initial positioning of the apparatus 20 in the nasal cavity and/or can assist in lodging the apparatus 20 in position in the nasal cavity. The apparatus 20 comprises a preformed shape. The preformed shape can assist in preventing adverse deformation and/or movement of the apparatus 20 when the apparatus is filled with fluid.

Branch 26 comprises a closed end 36 and branch 28 comprises a closed end 38. To facilitate flow of fluid into the respective sinuses, each of the branches (26,28) independently define a plurality of apertures 32 distributed along the body of each branch (26,28). The plurality of apertures 32 can facilitate a more effective distribution of fluid throughout the sinus. The apertures 32 provide a plurality of fluid outlets into a sinus and are shown in the enlarged view of a section of the branch 26 in FIG. 5a. In this embodiment, the apertures 32 comprise a circular shape. However, it will be appreciated that, in other embodiments, the apertures can comprise alternative shapes. For example, the branches (26,28) can define slit-like apertures 34, as shown in FIG. 5b. Other shapes of apertures include, diamond, triangular, square or other polygonal shape.

Whilst not shown in the Figures, in some embodiments, the apertures are transformable between an open state and a closed state. In such cases, where a positive pressure is applied to the apparatus, the apertures move into the open state and allow fluid flow out of the apparatus into a sinus. When the pressure is removed, the apertures move into the closed state and can prevent and/or limit backflow of fluid from a sinus into the apparatus through the apertures.

The relatively small size of the apertures 32 facilitates the delivery of irrigation fluid to a sinus in a jet-like manner which may assist in the flushing/irrigation of the sinuses.

In some embodiments, the body of the primary tube also defines a plurality of apertures to facilitate irrigation of the nasal passage as well as the sinuses.

In use, after insertion of the apparatus 20 into a nasal cavity of a patient, a fluid control system 25 is connected to the proximal end 24 of the primary tube 22. Irrigation fluid (e.g. a saline solution or water) held in a fluid reservoir within the fluid control system 25 is introduced into the apparatus 20 under pressure. The irrigation fluid flows through the primary tube 22 and the branches (26,28) and exits from the apparatus 20 through the apertures 32 into the sinuses. This flow of fluid is illustrated by the arrows on FIG. 3. The branches (26,28) assist in the flushing of a plurality of sinuses by supplying pressurised fluid directly into the sinuses.

In the embodiment shown in FIGS. 2-4, the apparatus 20 also provides a pathway for fluid removal from the sinuses, when suction is applied to the apparatus 20 by the fluid control system 25. Irrigation fluid can also be removed from the sinuses by drainage and, in some embodiments, this is the major pathway for fluid removal.

The apparatus 20 can be inserted into a nasal cavity of a patient, e.g. by a medical professional. The apparatus 20 can be left in position in a nasal cavity between irrigation treatments. For example, after initial irrigation of a sinus, the fluid control system 25 can be disconnected from the primary tube 22. After a predetermined time period, the fluid control system 25 can be reconnected to the primary tube 22 and a further irrigation treatment can be carried out. In this way, multiple irrigations of a sinus can be carried out using the same apparatus 20. For example, irrigations can be carried out on the same patient at regular time intervals over a predetermined period of time. For example, the apparatus can be left in position in a nasal cavity for a period between about 24 hours and about 48 hours. During this period, a patient can periodically connect the fluid control system 25 to the apparatus to facilitate periodic treatment of a sinus. This can enable multiple treatments of the sinus in the absence of a medical professional.

Further embodiments of the present invention will now be described with reference to FIGS. 6-9.

In the embodiment shown in FIG. 6, the apparatus 40 comprises a branch 48 extending into the right maxillary sinus 18a. Sub-branch 48a extends from the branch 48 at secondary junction region 49 into the sinus 18a. Sub-branch 48a extends in a different direction to branch 48 within the sinus 18a. Such a configuration may assist in irrigating a larger volume of the sinus as the sub-branch 48a and the branch 48 extend in different directions within the sinus 18a.

The apparatus 40 also comprises a branch 46 extending into the right frontal sinus 12a. In a similar fashion, sub-branches 46a and 46b extend from the branch 46 at secondary junction region 47 into the sinus 12a. In this embodiment, the sub-branches 46a and 46a are co-located at the same secondary junction region 47. However, it will be appreciated that in other embodiments, the sub-branches comprised on the same branch can extend from different secondary junction regions.

As will be appreciated from the foregoing, any number of sub-branches can extend from a branch, for example, 2, 3, 4, 5 or more sub-branches can extend from the same branch.

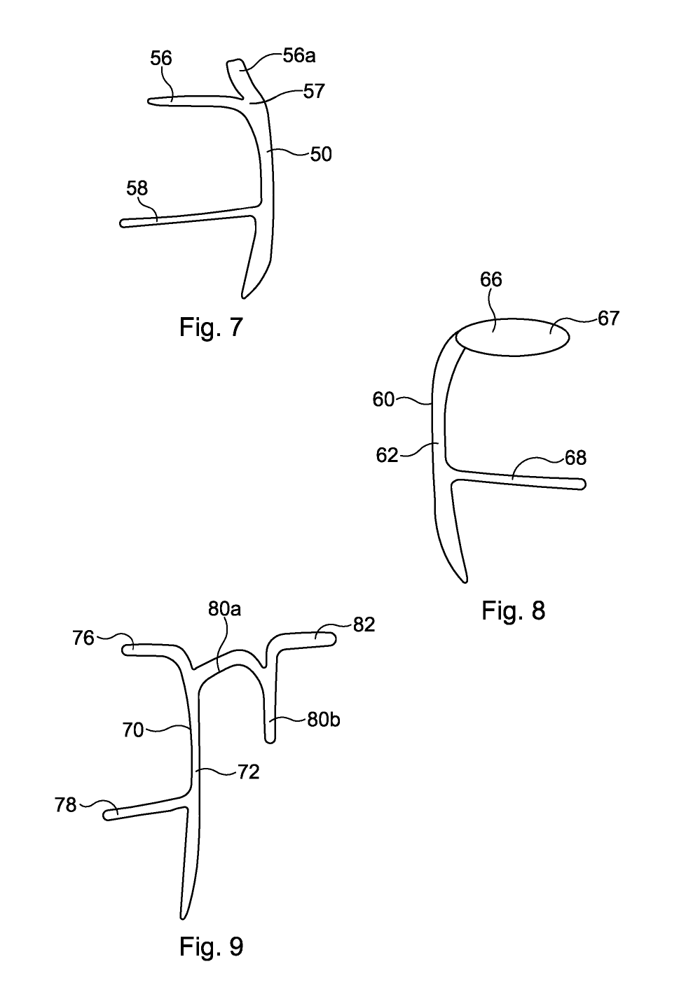

In the embodiment shown in FIG. 7, the apparatus 50 comprises a branch 58 configured to extend into a maxillary sinus and a branch 56 configured to extend into a frontal sinus. Sub-branch 56a extends from branch 56 at secondary junction region 57. Branch 56 and sub-branch 56a are positioned at an angle to one another. For instance in the embodiment shown in FIG. 7, branch 56 and sub-branch 56a are positioned at substantially 90° to one another.

In the embodiment shown in FIG. 8, the apparatus 60 comprises a primary tube 62 and a branch 68 extending from the body of the primary tube 62, which when positioned in a patient, is configured to extend into a maxillary sinus of a patient. The apparatus 60 further comprises a branch 66. Branch 66 is in the form of a balloon 67. Balloon 67 may be inflated to assist in the dilation of a sinus passageway and may assist in improving drainage from a sinus.

Whilst the embodiments shown in FIGS. 2-8 facilitate unilateral irrigation of a sinus or plurality of sinuses, e.g. irrigation of the sinuses on one side of a patient, in certain embodiments, the apparatus may facilitate bilateral irrigation, e.g. irrigation of sinuses on the right and left side of a patient simultaneously. One such embodiment is shown in FIG. 9.

The apparatus 70 comprises a primary tube 72. Branch 78 and branch 76 extend outwards from the body of the primary tube 72 and are configured respectively to extend into a right maxillary sinus and a right frontal sinus of a patient. The apparatus 70 also comprises a bridging portion (80a, 80b). The bridging portion (80a, 80b) is configured to extend between the left and right nasal passages. The bridging portion can act as a hook over the left and right nasal passages and can assist in lodging the apparatus 70 in position in a patient. The apparatus 70 further comprises a branch 82 which is configured to extend into a left frontal sinus of a patient. In this embodiment, after connection of a fluid control system, the apparatus 70 is configured to facilitate irrigation of the right maxillary sinus, and both frontal sinuses simultaneously.

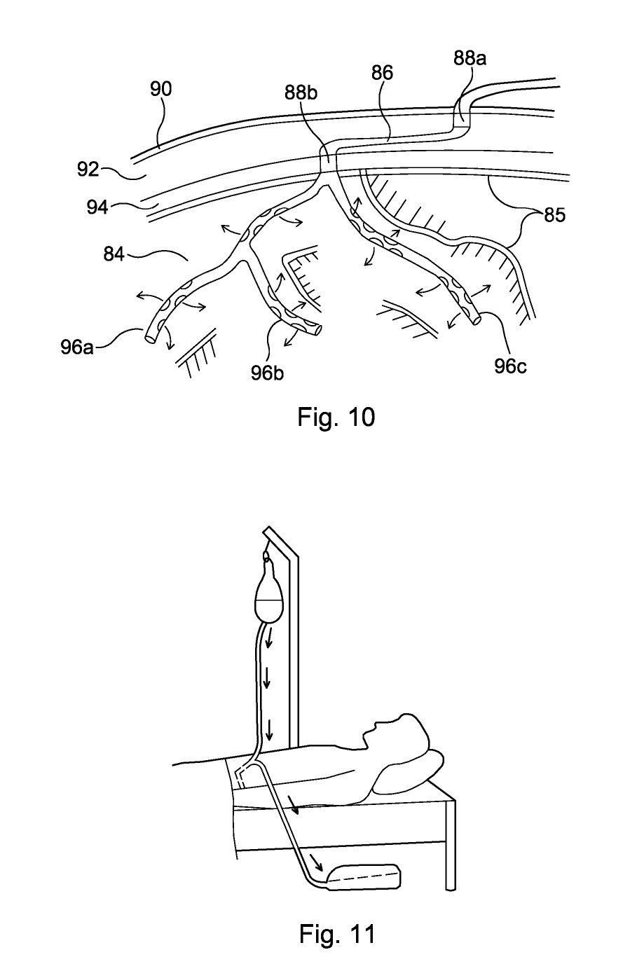

In an alternative embodiment, shown in FIG. 10, there is shown an apparatus 84 for treatment of a plurality of cavities in a peritoneal space defined by the peritoneum 85. The apparatus is configured to insert into peritoneal region via a catheter system 86 (e.g. a Tenckhoff catheter). A user can then insert the apparatus 84 into a peritoneal space through the skin 90, fat 92 and muscle 94 layers. In the embodiment shown, the catheter system is retained in positioned by Dacron cuffs 88a and 88b.

The apparatus 84 comprises a plurality of branches 96a, 96b and 96c which extend into different areas of a peritoneal space. The plurality of branches 96a, 96b and 96c each comprise a plurality of apertures to facilitate irrigation of different regions of a peritoneal space.

In use, the apparatus 84 can be used in the treatment of peritonitis by peritoneal lavage as shown schematically in FIG. 11.

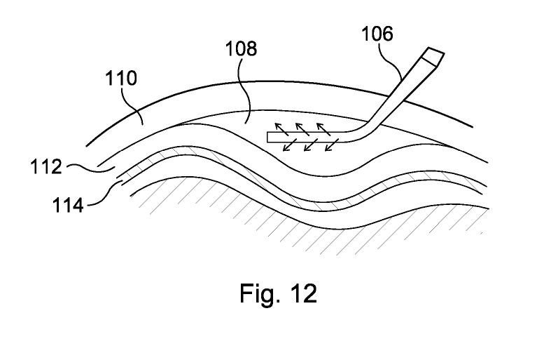

In an alternative embodiment, there is shown in FIG. 12 an apparatus 106 for treatment of an epidural abscess 108. The epidural abscess 108 is formed between the skull 110 and the dura mater layer 112. The apparatus 106 could equally be used in the treatment of a subdural abscess, for example an abscess formed between the dura mater and the underlying arachnoid layer 114.

The apparatus 106 can be inserted into the intracranial space by means known in the art. After insertion, fluid can be delivered to the abscess 108 as described in relation to the other embodiments. A plurality of apertures distributed along the apparatus 108 enable an effective delivery and distribution of fluid throughout the abscess space.

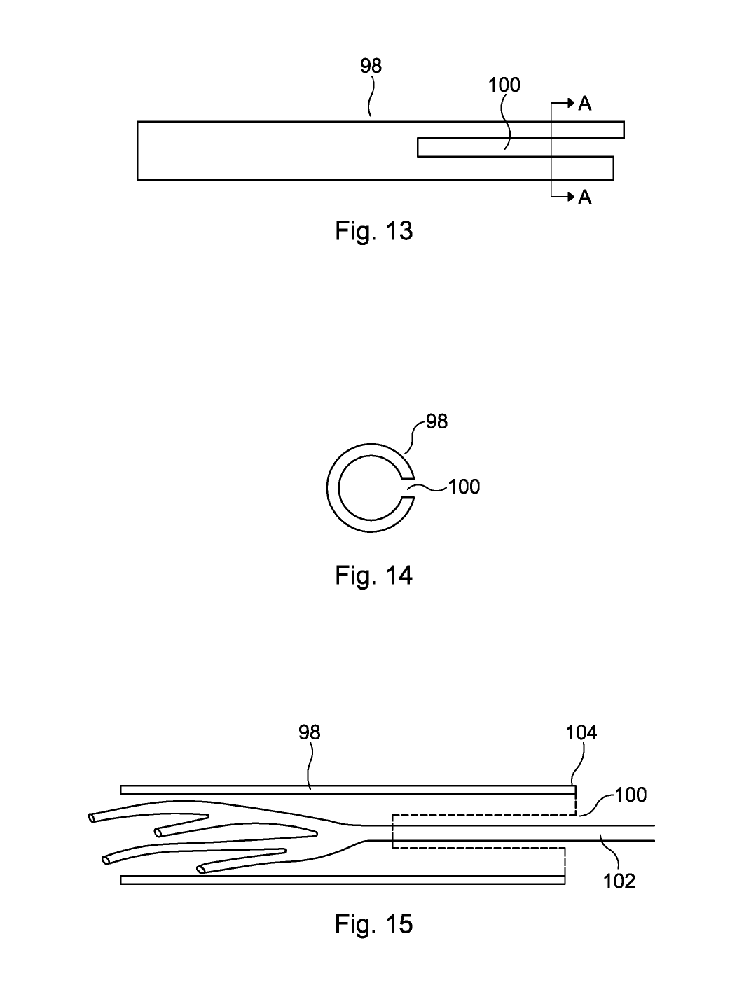

A deployment tube 98 (as shown in FIGS. 13-15) can be used to store an apparatus according to embodiments of the invention. The deployment tube comprises a tubular structure which defines a slot 100. The slot 100 extends partially along a length of the tubular structure from a first end. In alternative embodiments, the slot is coterminous with the tubular structure and extends along the entire length of the tubular structure. Alternatively, the slot may not be present on the deployment tube.

An apparatus 102 according to one embodiment of the invention is shown stored within a deployment tube 98 in FIG. 15. The apparatus is diametrically compacted within the deployment tube which can facilitate insertion of the apparatus into a body cavity, e.g. a nasal cavity.

The apparatus 102 can be slidingly removed from the deployment tube 98. In this instance the apparatus extends beyond the first end 104 of the deployment tube 98. This can facilitate extraction and manipulation of the apparatus. For example, a user can hold the apparatus 102 in position whilst slidingly removing the deployment tube 98.

In FIG. 15, the optional slot 100 is depicted with a broken line. When present, the slot can provide access for a deployment tool (not shown) to increase ease of deployment of the apparatus and/or removal of the deployment tube from the apparatus.

1. An apparatus for treatment of an anatomical region comprising:
a primary tube connectable to a fluid control system, and
at least one branch extending from the primary tube and in fluid communication with the primary tube,
wherein the apparatus is insertable into a body cavity and, when inserted, at least one branch extends into an anatomical region.

2. The apparatus according to claim 1 wherein the primary tube and/or at least one branch define an aperture or a plurality of apertures.

3. The apparatus according to claim 2 wherein the plurality of apertures are distributed along and/or around the body of the primary tube and/or at least one branch.

4. The apparatus according to claim 2 wherein at least one aperture defines a fluid outlet and/or fluid inlet.

5. The apparatus according to claim 2 wherein the aperture or apertures are transformable between an open and a closed state.

6. The apparatus according to claim 1 wherein the apparatus comprises a plurality of branches.

7. The apparatus according to claim 1, wherein the apparatus is configured to facilitate the treatment of a plurality of anatomical regions simultaneously.

8. The apparatus according to claim 1 wherein two or more of a plurality of branches are configured to extend into a separate or distinct anatomical region.

9. The apparatus according to claim 1 wherein the apparatus further comprises a fluid control system connected to the primary tube to supply fluid to an anatomical region and/or remove fluid from an anatomical region.

10. The apparatus according to claim 9 wherein at least one of the primary tube and/or the at least one branch provide a fluid pathway from the fluid control system to an anatomical region.

11. The apparatus according to claim 1 wherein at least one branch is configured to facilitate flushing of an anatomical region by supplying pressurised fluid directly into the anatomical region.

12. The apparatus according to claim 1 wherein the apparatus is configured to become pressurised when fluid is supplied to the primary tube.

13. The apparatus according to claim 1 wherein at least one branch extends transversely from the primary tube.

14. The apparatus according to claim 1 wherein the apparatus comprises at least one primary junction region.

15. The apparatus according to claim 14, wherein at least one primary junction region is located at an intermediate position between the distal and proximal ends of the primary tube.

16. The apparatus according to claim 1 wherein at least one branch comprises a fixed geometric arrangement with respect to the primary tube.

17. The apparatus according to claim 1 further comprising at least one sub-branch extending from at least one branch.

18. The apparatus according to claim 1 wherein the apparatus comprises at least one secondary junction region.

19. The apparatus according to claim 1 wherein a plurality of branches and/or sub-branches is configured to extend into a same anatomical region.

20. The apparatus according to claim 1 wherein a plurality of branches and/or sub-branches are configured to extend into separate anatomical regions.

21. The apparatus according to claim 1 further comprising a bridging portion.

22. The apparatus according to claim 1 wherein the apparatus comprises a flexible material and/or is resiliently deformable.

23. The apparatus according to claim 1 wherein the primary tube and at least one branch comprise a first and second component of a modular system.

24. The apparatus according to claim 1 wherein the apparatus is configured to facilitate circulation of fluid through and/or via an anatomical region or a plurality of anatomical regions.

25. The apparatus according to claim 1 wherein the apparatus is at least partially stored within a tubular structure.

26. The apparatus according to claim 25 wherein the tubular structure is a deployment tube.

27. The apparatus according to claim 1 wherein the anatomical region is a sinus.

28. The apparatus according to claim 1 wherein at least one branch is configured to extend into a frontal sinus and at least one branch is configured to extend into a maxillary sinus.

29. The apparatus according to claim 1 wherein the apparatus is configured to facilitate unilateral irrigation of a sinus or plurality of sinuses on one side of a patient.

30. The apparatus according to claim 1 wherein the apparatus is configured to facilitate bilateral irrigation of a plurality of sinuses.

31. The apparatus according to claim 1 wherein the apparatus is configured for treatment of a cavity in the peritoneal space e.g. paracolic gutters and subphrenic areas, a pleural cavity, a joint cavity, a bladder or an abscess space or the apparatus is configured for treatment of a cerebral ventricular space or an intracranial abscess.

32. An apparatus for treatment of an anatomical region comprising:
a primary tube,
at least one branch extending from the primary tube, and
a fluid control system connectable to the primary tube,
the primary tube being insertable into a body cavity and, when inserted, at least one branch extends into an anatomical region.

33. A kit for treatment of an anatomical region comprising the apparatus according to claim 1.

34. The kit according to claim 33, further comprising a plurality of primary tubes and/or a plurality of branches.

35. The kit according to claim 34 wherein the plurality of branches are configured for interchangeable attachment to the plurality of primary tubes.

36. A method for treatment of an anatomical region comprising:
inserting an apparatus into a body cavity of a patient, the apparatus comprising a primary tube, wherein the primary tube is in fluid communication with at least one branch configured to extend into an anatomical region,
connecting a fluid control system to the apparatus,
introducing fluid to the anatomical region and/or removing fluid from the anatomical region, wherein the apparatus provides a fluid pathway between the fluid control system and the anatomical region.

37. The method for treatment of an anatomical region according to claim 36 wherein the method comprises inserting the apparatus into a body cavity of a patient using a deployment tube.

38. The method for treatment of an anatomical region according to claim 36 wherein the fluid comprises a solution or dispersion comprising an active agent, or a plurality of active agents.

39. The method for treatment of an anatomical region according to claim 36 wherein the apparatus is left in position in the body cavity of a patient between treatments.

40. An apparatus for treatment of an anatomical region comprising:
a primary tube connectable to a fluid control system, the primary tube defining a plurality of apertures along a body of the primary tube,
wherein the apparatus is insertable into a body cavity.

41. (canceled)

42. The apparatus according to claim 5, wherein the aperture or apertures are transformable between the open and closed states upon at least one of pressurisation of the apparatus and a pressure differential being applied across the aperture or apertures.

43. The apparatus according to claim 14, wherein at least one primary junction region is located at the distal end of the primary tube.
